(19) United States
(12) Patent Application Publication
(10) Pub. No.: US 2026/0272643 A1
(43) Pub. Date: Sep. 17, 2026

(54) PROSTHETIC HEART VALVE WITH EXPANDABLE SKIRT TO PROMOTE WASHOUT

(71) Applicant: Edwards Lifesciences Corporation, Irvine, CA (US)

(72) Inventors: Brendan Michael Dalbow, Oceanside, CA (US); Justin Mark Nubla, Irvine, CA (US)

(73) Assignee: Edwards Lifesciences Corporation, Irvine, CA (US)

(21) Appl. No.: 19/668,636

(22) Filed: May 5, 2026

Related U.S. Application Data
(63) Continuation of application No. PCT/US2024/055629, filed on Nov. 13, 2024.
(60) Provisional application No. 63/599,859, filed on Nov. 16, 2023.

(51) Int. Cl. A61F 2/24

(52) U.S. Cl. CPC A61F 2/2418; A61F 2/2433; A61F 2210/0014; A61F 2220/0075; A61F 2250/0037; A61F 2250/006; A61F 2250/0069

(57) ABSTRACT

A prosthetic heart valve comprises a frame, multiple leaflets within the frame and a skirt assembly. The frame is expandable radially from a compressed state to an expanded state and comprises openings defined by struts. The skirt assembly is mounted to an outer surface of the frame and comprises a sealing layer and a plurality of expanding members. The sealing layer has an inflow and outflow edges secured to the frame and an intermediate portion between the edges. The expanding members displace the intermediate portion radially outward from the frame when the frame is expanded, thereby enlarging a neosinus between the leaflets and the sealing layer. When the prosthetic heart valve is implanted and the valve frame is expanded to the radially expanded state, retrograde blood inside the valve frame can flow outwardly through cells of the valve frame and into space between the valve frame and the sealing layer.

### CROSS-REFERENCE TO RELATED APPLICATIONS

This application is a continuation of PCT Application No. PCT/US2024/055629, filed November 13, 2024, which claims the benefit of U.S. Provisional Patent Application No. 63/599,859, filed November 16, 2023, wherein each of the above-referenced applications is incorporated by reference herein in its entirety.

### FIELD

The present disclosure relates to prosthetic heart valves, including frames for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (for example, through a femoral artery and the aorta) until the prosthetic valve reaches the implantation site in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

Most expandable, prosthetic heart valves comprise a radially expandable and compressible cylindrical metal (or wire) frame or stent and prosthetic leaflets mounted inside the frame. Commissures can be formed by connecting pairs of commissure tabs (or adjacent sides) of adjacent leaflets to each other and to struts of the frame (for example, commissure windows formed in the frame or to struts forming a cell of the frame). The prosthetic leaflets mounted inside the frame are designed to open during systole (spreading away from each other) and to close during diastole (collapsing toward each other). An outer skirt or sealing member is generally positioned on the outside of the frame for positioning between the frame and the native leaflets when the prosthetic valve is expanded at the implantation site to promote sealing of the prosthetic heart valve with the native anatomy. Some prosthetic heart valves may also include an inner skirt mounted to the inner surface of the frame.

An annular region between the outer skirt (or the inner skirt) and each of the prosthetic leaflets defines a neosinus that can have a restricted volume near its bottom wherein the leaflets are coupled to the frame.

### SUMMARY

Described herein are prosthetic heart valves, delivery apparatuses, and methods for implanting prosthetic heart valves. The disclosed prosthetic heart valves, delivery apparatus, and methods can, for example, help promote washout. As such, the devices and methods disclosed herein can, among other things, overcome one or more of the deficiencies of typical prosthetic heart valves and their delivery apparatus.

A prosthetic heart valve can comprise a frame and a valvular structure coupled to the frame. In addition to these components, a prosthetic heart valve can further comprise one or more of the components disclosed herein.

In some examples, a prosthetic heart valve can comprise a sealing member or skirt assembly configured to reduce paravalvular leakage.

In some examples, a prosthetic heart valve can comprise a skirt assembly with expanding members configured to displace an intermediate portion thereof radially outward from the valve frame when the prosthetic heart valve is in its radially expanded state, thereby enlarging the neosinus.

In some examples, a prosthetic heart valve can comprise a skirt assembly configured to be displaced radially outward from the valve frame when the prosthetic heart valve is in its radially expanded state, thereby enlarging the neosinus.

In some examples, a prosthetic heart valve assembly can comprise a docking component with a sealing layer or skirt configured to be implanted its radially expanded state, a prosthetic heart valve component configured to be implanted in the docking component and a neosinus defined between the leaflets and the sealing layer.

In some examples, a prosthetic heart valve comprises:

a valve frame comprising an outflow end and an inflow end, wherein the valve frame is radially expandable from a radially compressed state to a radially expanded state, wherein the valve frame comprises a plurality of struts arranged to form a plurality of cells defining openings in the valve frame;

a plurality of leaflets disposed within and coupled to the valve frame; and

a skirt assembly mounted to an outer surface of the valve frame, wherein the skirt assembly comprises a sealing layer and a plurality of expanding members coupled to the sealing layer, wherein the sealing layer has an inflow edge and an outflow edge secured to the valve frame and an intermediate portion between the inflow edge and the outflow edge, wherein the expanding members are configured to displace the intermediate portion radially outward from the valve frame when the valve frame is expanded to the radially expanded state to enlarge a neosinus defined between the leaflets and the sealing layer;

wherein when the prosthetic heart valve is implanted in a native heart valve and the valve frame is expanded to the radially expanded state, retrograde blood inside the valve frame can flow outwardly through cells of the valve frame and into a region of the neosinus that is between the valve frame and the sealing layer.

In some examples, a prosthetic heart valve comprises:

a valve frame comprising an outflow end and an inflow end, wherein the valve frame is radially expandable from a radially compressed state to a radially expanded state, wherein the valve frame comprises a plurality of struts arranged to form a plurality of cells defining openings in the valve frame;

a plurality of prosthetic leaflets disposed within and coupled to the valve frame; and

a skirt assembly mounted to an outer surface of the valve frame, wherein the skirt assembly comprises a sealing layer having an inflow edge and an outflow edge, wherein a portion of the sealing layer is configured to be displaced radially outward from the valve frame when the valve frame is expanded to the radially expanded state to enlarge a neosinus defined between the prosthetic leaflets and the sealing layer;

wherein when the prosthetic heart valve is implanted in a native heart valve and the valve frame is expanded to the radially expanded state, retrograde blood inside the valve frame can flow outwardly through cells of the valve frame and into a region of the neosinus that is between the valve frame and the sealing layer.

In some examples, a prosthetic heart valve comprises one or more of the components recited in Examples 1-13 below.

In some examples, a prosthetic heart valve assembly comprises:

a docking component that is radially expandable from a radially compressed state to a radially expanded state, the docking component comprising an annular docking frame comprising an inflow end, an outflow end, and an intermediate portion extending axially between the inflow end and the outflow end, wherein a diameter of the intermediate portion is greater than a diameter of the inflow end and a diameter of the outflow end in the radially expanded state, the docking component further comprising an annular sealing layer covering a surface of the docking frame; and

a prosthetic heart valve component, separate from the docking component, that is radially expandable from a radially compressed state to a radially expanded state, the prosthetic heart valve component comprising an annular valve frame and a plurality of leaflets disposed within and coupled to the valve frame, wherein the valve frame comprises a plurality of struts arranged to form a plurality of cells defining openings in the valve frame;

wherein the docking component is configured to be implanted in a native heart valve in its radially expanded state and the prosthetic heart valve component is configured to be implanted in the docking component in a radially expanded state such that a neosinus is defined between the leaflets and the sealing layer that allows retrograde blood inside the valve frame to flow outwardly through cells of the valve frame and into space between the valve frame and the sealing layer.

In some examples, a prosthetic heart valve assembly comprises one or more of the components recited in Examples 14-20 below.

In some examples, a method of implanting a prosthetic heart valve and a docking component with a delivery system, comprises:

positioning the docking component at a docking component position on the delivery system, the docking component having an expandable docking component frame and a skirt coupled to an outside of the docking component frame;

positioning the prosthetic heart valve at a prosthetic heart valve position;

advancing the delivery system to an implantation site to align the docking component with a native valve annulus;

radially expanding the docking component frame;

further radially expanding the skirt coupled to the docking component frame against native leaflets;

moving the delivery system to align the prosthetic heart valve with the docking component; and

radially expanding the prosthetic heart valve into contact with the docking component, wherein an enlarged neosinus is defined between an outside of the prosthetic valve and an inside of the skirt of the docking component, the enlarged neosinus promoting increased washout of blood.

In some examples, a method of implanting a prosthetic heart valve includes one or more of the steps recited in Example 21. below.

The above method(s) can be performed on a living animal or on a simulation, such as on a cadaver, cadaver heart, anthropomorphic ghost, simulator (for example, with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2 is a side view of an exemplary delivery apparatus configured to deliver and implant a radially expandable prosthetic heart valve at an implantation site.

FIG. 3 is a side sectional view of a prosthetic heart valve as implanted in a patient and showing a neosinus defined between leaflets of the prosthetic heart valve and a skirt of the prosthetic heart valve.

FIG. 4A is an additional side sectional view showing the prosthetic heart valve leaflets, frame, neosinus and native leaflets relative to a direction of blood flow through the prosthetic heart valve.

FIG. 4B is a cross-sectional view of the prosthetic heart valve leaflets, frame, neosinus and native leaflets (shown at a different rotation relative to FIG. 4A), with the leaflets of the prosthetic heart valve shown in an open position for one leaflet and in a closed position for the remaining two leaflets.

FIG. 5 is a simplified side view of the right side of FIG. 4A, showing a minimal flow area between the leaflet of the prosthetic heart valve, and the frame/skirt positioned radially outward thereof, that can result in reduced washout.

FIGS. 6 and 7 are simplified side views similar to FIG. 5, except showing a new frame and skirt arrangement having a bulb shape when expanded (FIG. 7) to promote increased washout.

FIG. 8 is side view of the new frame and skirt arrangement showing expanding members in or next to the skirt.

FIG. 9 is a side view of the skirt with expanding members shown in an expanded state.

FIG. 10A is a simplified side view of a prosthetic heart valve with the new frame and skirt arrangement shown in a non-expanded state.

FIG. 10B is a simplified side view of a prosthetic heart valve with the new frame and skirt arrangement shown in an expanded state with the expanding members urging the skirt into the bulb shape.

FIG. 11 is a simplified side view similar to FIG. 5, except showing the skirt expanded to have the bulb shape.

FIG. 12 is a perspective view of a docking component and a docking component sealing element implantable configured to urge native leaflets radially outward to promote washout.

FIG. 13 is a simplified side view of the docking component and docking component sealing element showing the bulb shape in the expanded state.

FIG. 14 is a perspective view of the docking component and sutures extending from a frame of a prosthetic heart valve to the docking component.

FIGS. 15A and 15B are side sectional views of a representative delivery system for the docking component and prosthetic heart valve at different stages in an implantation procedure.

FIG. 16 is a simplified side sectional view showing the docking component after it has been deployed from the delivery system and expanded.

FIG. 17 is a simplified side sectional view showing the prosthetic heart valve being expanded before it is deployed from the delivery system.

FIG. 18 is a simplified side sectional view showing the prosthetic heart valve after implantation and the delivery system being withdrawn.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient’s body), while distal motion of the device is motion of the device away from the user and toward the implantation site (for example, into the patient’s body). The terms “longitudinal” and “axial” refer to an axis extending in the proximal and distal directions, unless otherwise expressly defined.

As used herein, “e.g.” means “for example,” and “i.e.” means “that is.”

### Overview of the Disclosed Technology

As introduced above, prosthetic heart valves can include prosthetic leaflets that are secured inside the frame laterally or radially opposite a skirt that surrounds the outside of the frame. A region between the leaflets (located inside the frame) and a skirt (located inside or outside the frame) is referred to as a neosinus. The leaflets and skirt are generally solid structures that do not allow blood to flow past (the frame positioned between the leaflets and the skirt has openings and thus does not generally restrict blood flow). Blood flow in the neosinus can be limited, especially where the neosinus has a reduced cross-sectional area, such as near a bottom of the neosinus where the leaflet is coupled to the frame with only a minimal distance separating the leaflet from the skirt.

Described herein are various examples of a skirt assembly having expanding members configurable to expand the skirt radially outward to enlarge the neosinus and promote greater washout of blood.

In some examples, the skirt is coupled to the frame and has expanding members (for example, shape-memory members) that can be expanded when the prosthetic heart valve is in the implantation site. The expanding members urge the skirt radially outward to enlarge the neosinus. The expanded skirt can have a bulb-shaped cross-section.

In some examples, the skirt (also sometimes referred to herein as the sealing element) is coupled to a docking component (also sometime referred to herein as a dock) that is separate from the prosthetic heart valve. The docking component is delivered separately in advance of delivery of the prosthetic heart valve. The docking component is expanded from a crimped state to its expanded state. Expanding members are configured to urge the skirt of the docking component radially outward to enlarge the neosinus. The expanded skirt can have a bulb-shaped cross-section.

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient’s vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

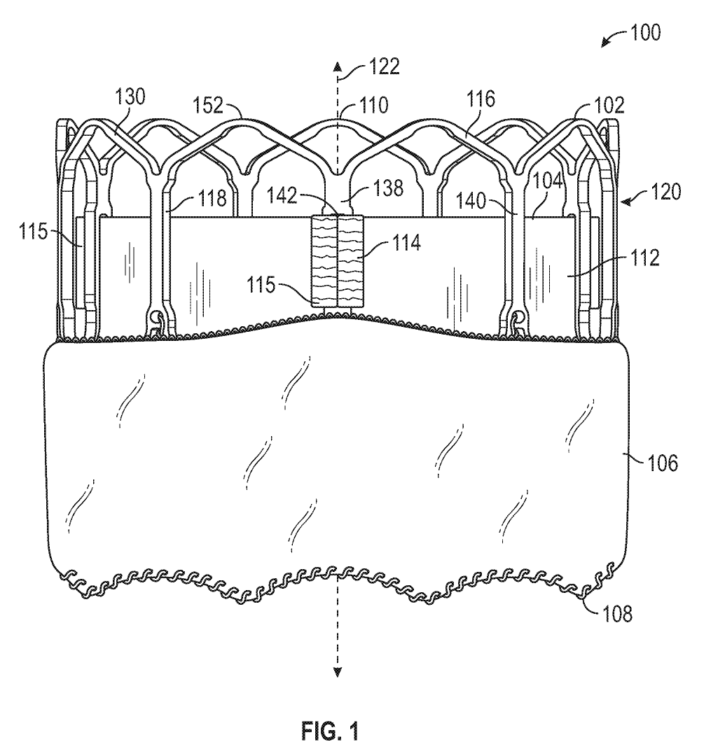

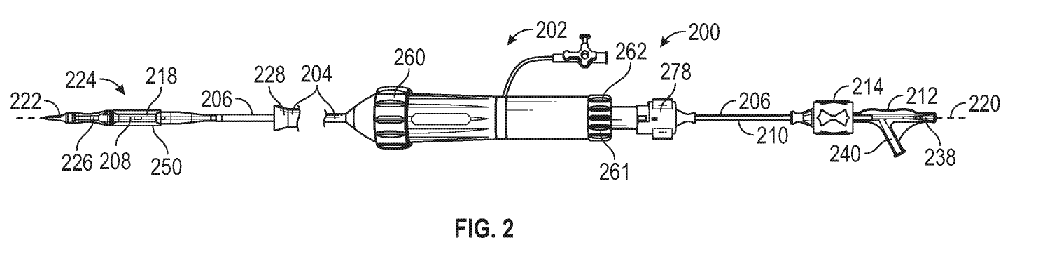

FIG. 1 illustrates an exemplary prosthetic device (for example, prosthetic heart valve) comprising a frame, leaflets secured on an inside of the frame, and an outer skirt disposed around an outer surface of the frame. In some examples, the frame can comprise commissure windows configured to receive adjacent sides of adjacent leaflets, thereby forming a commissures that are secured to the frame. The prosthetic device can be advanced through a patient’s vasculature, such as to a native heart valve, by a delivery apparatus, such as the exemplary delivery apparatus shown in FIG. 2.

### Examples of the Disclosed Technology

FIG. 1 shows a prosthetic heart valve 100 (prosthetic valve), according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated herein by reference. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

The prosthetic heart valve 100 can include a stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102.

The valvular structure 104 can comprise a plurality of leaflets 112 (for example, three leaflets, as shown in FIG. 1), collectively forming a leaflet structure, which can be arranged to collapse in a tricuspid arrangement (or bicuspid arrangement in some examples). The leaflets 112 can be secured to one another at their adjacent sides (for example, commissure tabs) to form commissures 114 of the valvular structure 104. For example, each leaflet 112 can comprise opposing commissure tabs disposed on opposite sides of the leaflet 112 and a cusp edge portion extending between the opposing commissure tabs. The cusp edge portion of the leaflets 112 can have an undulating, curved scalloped shape, and can be secured directly to the frame 102 (for example, by sutures). However, in alternate examples, the cusp edge portion of the leaflets 112 can be secured to an inner skirt which is then secured to the frame 102. In some examples, the leaflets 112 can be formed of pericardial tissue (for example, bovine pericardial tissue), biocompatible synthetic materials, or various other suitable natural or synthetic materials as known in the art and described in U.S. Patent No. 6,730,118, which is incorporated by reference herein.

In some examples, the outer skirt 106 can be an annular skirt. In some instances, the outer skirt 106 can comprise one or more skirt portions that are connected together and/or individually connected to the frame 102. The outer skirt 106 can comprise a fabric or polymeric material, such as ePTFE, PTFE, PET, TPU, UHMWPE, PEEK, PE, etc. In some instances, instead of having a relatively straight upper edge portion, as shown in FIG. 1, the outer skirt 106 can have an undulating upper edge portion that extends along and is secured to the angled struts 130. Examples of such outer skirts, as well as various other outer skirts, that can be used with the frame 102 can be found in U.S. provisional patent application No. 63/366,599 filed June 17, 2022, which is incorporated by reference herein.

The frame 102 can be radially compressible and expandable between a radially compressed (or collapsed) configuration and a radially expanded configuration (the expanded configuration is shown in FIG. 1).

The frame 102 can be made of any of various suitable plastically-expandable materials (for example, stainless steel, etc.) or self-expanding materials (for example, Nitinol). When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIG. 1, the frame 102 can comprise a plurality of interconnected struts 116 which form multiple rows of open cells 118 between the outflow end 110 and the inflow end 108 of the frame 102. In some examples, the frame 102 can comprise three rows of cells 118 with a first (upper in the orientation shown in FIG. 1) row of cells 120 disposed at the outflow end 110. The first row of cells 120 comprises cells 118 that are elongated in an axial direction (relative to a central longitudinal axis 122 of the frame 102), as compared to cells 118 in the remaining rows of cells. For example, the cells 118 of the first row of cells 120 can have a longer axial length than cells 118 in the remaining rows of cells.

In some examples, as shown in FIG. 1, each row of cells comprises nine cells 118. Thus, in such examples, the frame 102 can be referred to as a nine-cell frame.

In alternate examples, the frame 102 can comprise more than three rows of cells (for example, four or five) and/or more or less than nine cells per row. In some examples, the cells 118 in the first row of cells 120 may not be elongated compared to cells 118 in the remaining rows of cells of the frame 102.

The interconnected struts 116 can include a plurality of angled struts 130 arranged in a plurality of rows of circumferentially extending rows of angled struts, with the rows being arrayed along the length of the frame 102 between the outflow end 110 and the inflow end 108. The interconnected struts 116 can also include a plurality of axially extending window struts 138 (or window strut portions) and a plurality of axial (or axially extending) struts 140. The axially extending window struts 138 (which can also be referred to as axial struts that include a commissure window) define commissure windows (for example, open windows) 142 that are spaced apart from one another around the frame 102, in a circumferential direction, and which are adapted to receive a pair of commissure tabs of a pair of adjacent leaflets 112 arranged into a commissure (for example, commissure 114). In some examples, the commissure windows 142 and/or the axially extending window struts 138 defining the commissure windows 142 can be referred to herein as commissure features or commissure supports, each commissure feature or support configured to receive and/or be secured to a pair of commissure tabs of a pair of adjacent leaflets.

One or more (for example, two, as shown in FIG. 1) axial struts 140 can be positioned between, in the circumferential direction, two commissure windows 142 formed by the window struts 138. Since the frame 102 can include fewer cells per row (for example, nine) and fewer axial struts 140 between each commissure window 142, as compared to some more traditional prosthetic heart valves, each cell 118 can have an increased width (in the circumferential direction), thereby providing a larger opening for blood flow and/or coronary access.

Each axial strut 140 and each window strut 138 forms an axial side of two adjacent cells of the first row of cells 120.

Commissure tabs 115 of adjacent leaflets 112 can be secured together to form commissures 114 (FIG. 1). Each commissure 114 of the prosthetic heart valve 100 comprises two commissure tabs 115 paired together, one from each of two adjacent leaflets 112, and extending through a commissure window 142 of the frame 102. Each commissure 114 can be secured to the window struts 138 forming the commissure window 142.

The cusp edge portion (for example, scallop edge) of each leaflet 112 can be secured to the frame 102 via one or more fasteners (for example, sutures). In some examples, the cusp edge portion of each leaflet 112 can be secured directly to the struts of the frame 102.

In some examples, the cusp edge portion of the leaflets 112 can be secured to an inner skirt and the inner skirt can then be secured directly to the frame 102.

Various methods for securing the leaflets 112 to a frame, such as the frame 102, are disclosed in U.S. provisional patent applications 63/278,922, filed November 12, 2021, and 63/300,302, filed January 18, 2022, both of which are incorporated by reference herein.

The frame 102 can further comprise a plurality of apex regions 152 formed at the inflow end 108 and the outflow end 110, each apex region 152 extending and forming a junction between two angled struts 130 at the inflow end 108 or outflow end 110. As such, the apex regions 152 are spaced apart from one another, in a circumferential direction at the inflow end 108 and the outflow end 110. Additional details and examples of frames for prosthetic heart valves that include apex regions can be found in PCT Application No. PCT/US2022/025687, which is incorporated by reference herein.

FIG. 2 shows a delivery apparatus 200, according to an example, that can be used to implant an expandable prosthetic heart valve (for example, the prosthetic heart valve 100 of FIG. 1 and/or any of the other prosthetic heart valves described herein). In some examples, the delivery apparatus 200 is specifically adapted for use in introducing a prosthetic valve into a heart.

The delivery apparatus 200 in the illustrated example of FIG. 2 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (for example, move) longitudinally, along a central longitudinal axis 220 of the delivery apparatus 200, relative to one another to facilitate delivery and positioning of a prosthetic valve at an implantation site in a patient’s body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (for example, inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222 (as shown in FIG. 2), or to an alternate component at the distal end of the delivery apparatus 200 (for example, a distal shoulder). An intermediate portion of the balloon 218 can overlay a valve mounting portion 224 of a distal end portion of the delivery apparatus 200 and a distal end portion of the balloon 218 can overly a distal shoulder 226 of the delivery apparatus 200. The valve mounting portion 224 and the intermediate portion of the balloon 218 can be configured to receive a prosthetic heart valve in a radially compressed state. For example, as shown schematically in FIG. 2, a prosthetic heart valve 250 (which can be one of the prosthetic valves described herein) can be mounted around the balloon 218, at the valve mounting portion 224 of the delivery apparatus 200.

The balloon shoulder assembly, including the distal shoulder 226, is configured to maintain the prosthetic heart valve 250 (or other medical device) at a fixed position on the balloon 218 during delivery through the patient’s vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic valve 250 is mounted in the radially compressed state on the valve mounting portion 224 (as shown in FIG. 2) and during delivery of the prosthetic valve to the target implantation site. As such, the distal tip portion 228 can be configured to resist movement of the prosthetic valve 250 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Patent No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (for example, for fine positioning at the implantation site). Further details on the delivery apparatus 200 can be found in PCT Application No. PCT/US2021/047056, which is incorporated by reference herein.

As introduced above and shown in FIG. 1, prosthetic valves can include leaflets (for example, leaflets 112) that are secured to one another at their adjacent sides (for example, commissure tabs) to form commissures (for example, commissures 114 in FIG. 1) which are secured to a frame of the prosthetic valve. In some examples, as shown in FIG. 1, the commissures can extend through commissure windows in the frame of the prosthetic heart valve and then be secured to the frame struts forming the commissures windows. The commissures may be static and unable to move freely during operation of the prosthetic valve (for example, when implanted in a native heart valve).

One challenge with prosthetic heart valves is that thrombosis can arise. According to Virchow’s triad, primary contributing factors to thrombosis are blood flow stasis (venous stasis), vascular injury (endothelial injury) and hypercoagulability. Prosthetic heart valves create a neosinus extending between the leaflets of the prosthetic heart valve (on the radially inward side) and the skirt (on the radial outward side). This neosinus region leads to stasis and inhibiting washout of stagnant blood from a region near the bottom cusp of the leaflets and the adjacent skirt.

Described herein are various examples of a skirt or skirt assembly having expanding members configurable to expand the skirt radially outward relative to the frame to enlarge the neosinus and promote greater washout of blood. The expanding members can be part of or arranged next to a skirt that is coupled to the frame of the prosthetic heart valve. Alternatively, there can be a docking component separate from prosthetic heart valve that has a skirt and expanding members. In both cases, the expanded skirt can have a bulb-shaped cross-section.

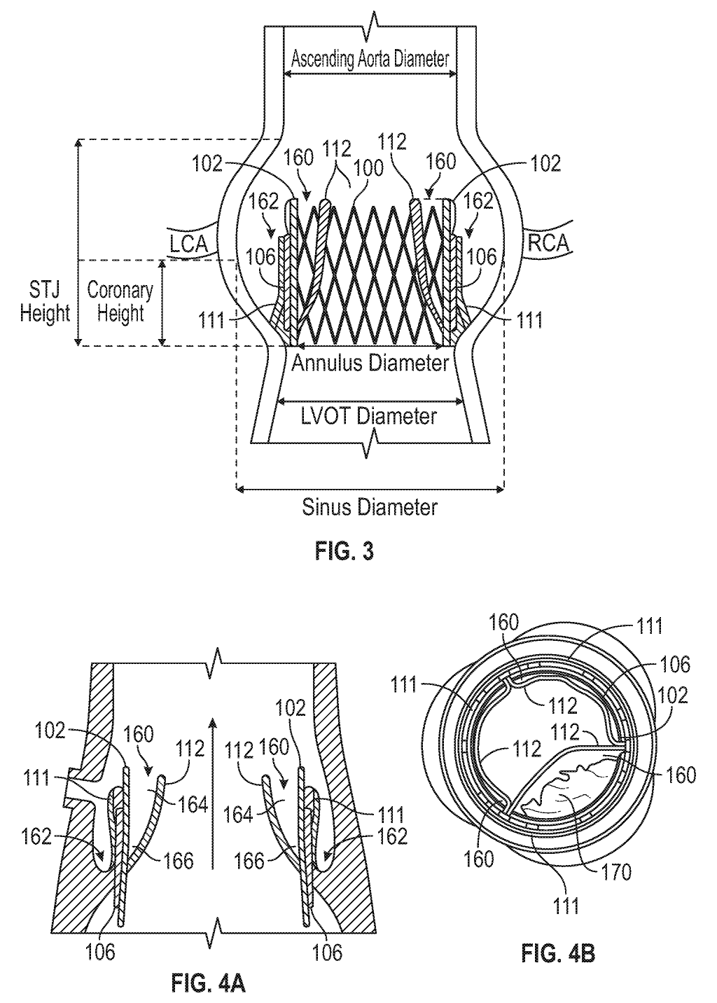

A neosinus 160 formed by a prosthetic heart valve 100 implanted in an annulus is shown in FIGS. 3 and 4A, which are two different exemplary illustrations. The neosinus 160 is defined as an area between the leaflets 112 of the prosthetic heart valve 100, at the radially inward side, and the frame 102/skirt 106 of the prosthetic valve 100 at the radially outward side. Overall, the neosinus 160 has a tapering shape with curved sides in cross-section, and tapers from its upper open end 164 (at the outflow end of the leaflets) to its bottom 166 at a closed lower end (at the inflow end of the leaflets). When implanted as shown, the prosthetic heart valve 100 contacts native leaflets 111 and urges them radially outward. An anatomic sinus 162 is defined between the native leaflets 111 and an outwardly adjacent vessel wall.

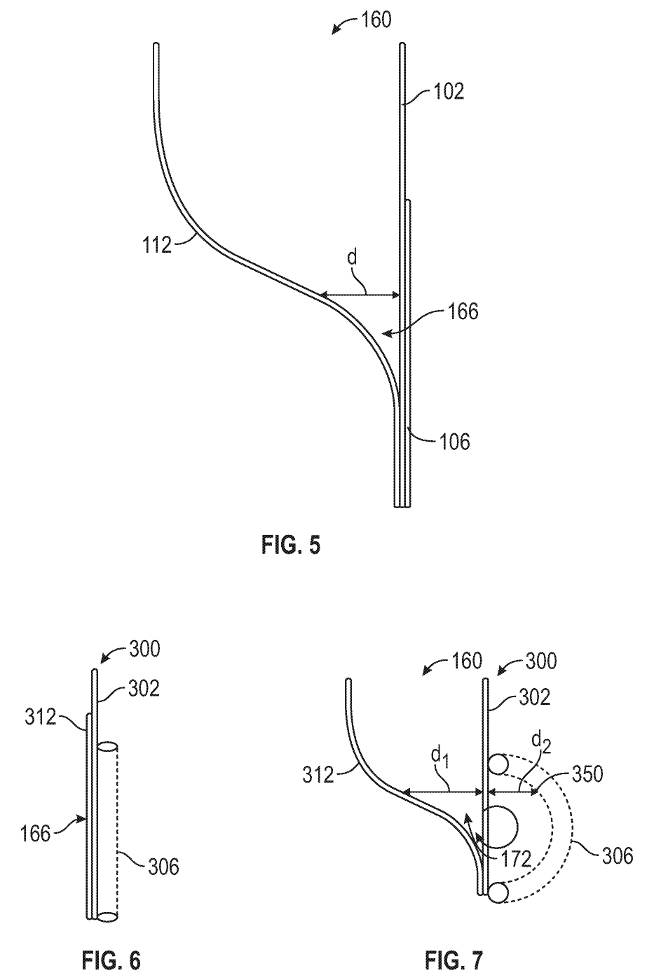

Stasis is more pronounced in the neosinus 160 than in the anatomical sinus 162 because the area of the neosinus 160 near the bottom 166 is comparatively much smaller. FIG. 5 is a simplified view of the right side of FIG. 3 or FIG. 4A showing the cross-section of the neosinus 160 and a resulting small radial dimension d of its open area near the bottom 166, which leads to minimal blood flow in that region. As illustrated, at the bottom 166, the leaflet 112 is very closely spaced to the skirt 106. The leaflet 112 and the skirt 106 have generally solid surfaces that tend to trap blood flow, whereas the frame 102 has openings tending to allow blood to flow. FIG. 4B depicts thrombosis 170 (blood clot) that can form within the neosinus 160 caused by inefficient washout in that area. As shown, the blood clot can prevent one of the prosthetic leaflets 112 from fully opening under the forward flow of blood.

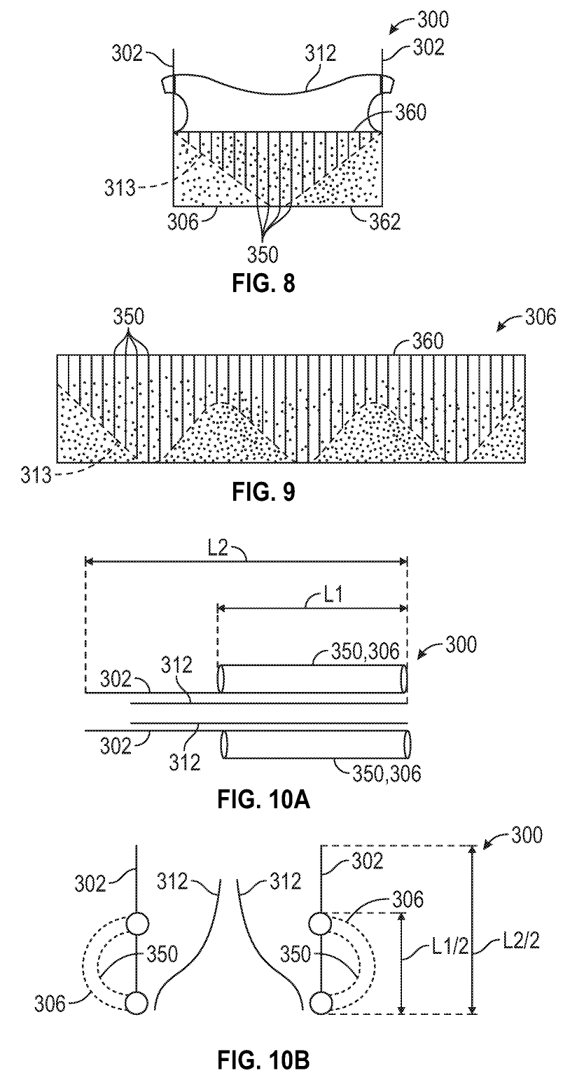

Referring to FIGS. 6 and 7, in a new prosthetic valve 300, a skirt 306 (also referred to as a sealing layer) is urged radially outwardly from its initial position against a frame 302 to a deployed position away from the frame when the prosthetic valve is radially expanded from its radially compressed state (FIG. 6) to a radially expanded state, thereby enlarging the neosinus 160 and promoting washout of blood (FIG. 7). FIG. 7 is a simplified side view of the new prosthetic valve 300, showing the right side that generally corresponds to FIG. 5, except the radial dimension of the open area (d₁and d₂) can be greater than the radial dimension d. The frame 302 (shown schematically in the figures) can be the same as the frame 102 or it can have various other configurations. The frame 302 can be made from a plastically expandable material or a self-expanding, shape memory material, as previously described with respect to the frame 102.

As shown, the prosthetic valve 300 has a skirt 306 that is radially expandable (FIG. 7) into a bulb shape to provide increased volume and promote washout, represented by arrow 172. The neosinus 160 is the space defined or bounded by the outer surfaces of the leaflets 312 and the inner surface of the skirt 306, which is spaced outwardly from the frame. In this manner, when the leaflets 312 are closed (such as during diastole), retrograde blood flows into the frame behind the leaflets, and then outwardly through the frame 302 into the space between the frame and the skirt, enhancing washout in the neosinus 160.

In some examples, the prosthetic valve 300 or the skirt 306 has expanding members to assist in urging the skirt radially outward. For example, expanding members 350 can be coupled to the skirt 306, or can be positioned between the frame 302 and the skirt 306. The expanding members can be coupled to the skirt at one or both of their ends, or at one or more portions long their lengths. FIGS. 6 and 7 illustrate an example where the expanding members 350 are positioned between the frame 302 and the skirt 306. As shown in FIG. 7, the expanding members 350 have been configured to contact an inner surface of the skirt 306 and urge it radially outward into a bulb shape as shown. The expanding members 350 assume an arc shape when the frame 302 is radially expanded, and the skirt 306 follows the arc shape. As indicated, the bottom 166 of the sinus 160 is expanded and has increased flow and washout.

FIGS. 8 and 9 illustrate another example where the expanding members 350 are incorporated into the skirt 306. For example, the expanding members 350 may be interwoven into the material of the skirt 306, such as by weaving the expanding members with the yarns forming the skirt. In some implementations, the expanding members 350 are described as elongate or as strands because they are relatively small in cross section and long.

FIG. 8 is a simplified side view of the prosthetic valve 300 in its expanded state showing the skirt 306 and leaflets 312 relative to the frame 302. The expanding members 350 can be positioned as shown to extend generally axially. The expanding members 350 can be positioned to have first ends (upper ends) at an outflow end 360 of the skirt 306, and second ends (lower ends) spaced in the axial direction away from the first ends and towards the inflow end 362. The expanding members 350 can have varying lengths.

The expanding members 350 can be arranged and sized such that their lower ends follow the shape of the cusps of the leaflets, such is shown for a lower end boundary 313 that follows an inflow edge boundary of the leaflet 312 in FIG. 8. In this manner, when the expanding members 350 urge the skirt 306 to its deployed position, the skirt has a generally tri-lobular shape that mimics the shape of a native heart valve sinus (for the example, the sinus of the native aortic valve). In some examples, skirt 306 can be stitched or otherwise secured to the frame 302 along the boundary 313 such that the portion of the skirt upstream of the boundary 313 does not necessarily protrude outwardly from the frame and instead can lie against the frame when the prosthetic valve is in its radially expanded state.

FIG. 9 shows the skirt 306 detached from the frame and extended flat. As illustrated, the lower end boundary 313 formed by the second or lower ends of the expanding members 350 defines a repeating curve that follows the shape of the three leaflet cusps. In some examples, the boundary 313 extends along and is aligned with the attachment of the cusp edges of the leaflets 312 to the frame 302.

The expanding members can be formed of a shape-memory material (also described above as a self-expanding material), for example, Nitinol or other similar materials. In this way, the expanding members can be designed to return to a predetermined shape upon being subjected to selected predetermined conditions.

FIG. 10A illustrates a simplified cross-section of the prosthetic valve 300 in a non-expanded state (for example, a crimped state) prior to implantation. The skirt 306 is fit closely around the frame 302 to minimize the overall diameter or dimension of the prosthetic valve 300. The expanding members 350, are contained within or positioned next to the skirt 306. The opposing ends of each expanding member optionally can be secured or fixed to the frame. For example, the ends of each expanding member can be secured (such as by welding) to adjacent struts of the frame. The skirt 306 can be stitched or otherwise secured to the frame along the outflow edge 360, the inflow edge 362, and/or along the boundary 313. FIG. 10B illustrates a simplified cross-section of the prosthetic valve 300 after the frame 302 has been expanded and the expanding members 350 have been expanded to urge the skirt 306 radially outward into the bulb shape as shown.

For purposes of illustration as shown in FIG. 10A, the frame 302 in its unexpanded state can be assumed to have a length L2, and the skirt 306 in its unexpanded state can be assumed to have a length L1. As described in greater detail below, radially expanding the frame 302 can cause the length to decrease from L2 to a shorter length, such as ½×L2 as shown in FIG. 10B. Similarly, expanding the expanding members 350 to urge the skirt 306 outward can cause the length of the skirt to decrease from L1 to ½×L1. If the opposing ends of the expanding members are fixed to the frame, foreshortening of the frame between the ends of the expanding members when the frame is radially expanded can cause the expanding members to buckle outwardly and help urge the skirt to its deployed state. In lieu of or in addition to fixing the ends of the expanding members to the frame, the expanding members can be made from a shape memory material (for example, Nitinol) and are shape set to assume their radially expanded state shown in FIG. 10B when released from a constraining force. For example, when the prosthetic valve is being delivered through the patient’s vasculature, a delivery capsule can extend over the prosthetic valve or a lasso can extend around the skirt. Deploying the prosthetic valve from the capsule or removing or loosening the lasso allows the shape-memory, self-expanding expanding members to revert to the expanded state shown in FIG. 10B.

In some examples, in lieu of or in addition to the expanding members 350, the skirt 306 can be shape set to expand away from the frame and assume the radially expanded shape, such as shown in FIG. 10B, when the prosthetic valve is radially expanded. For example, the skirt 306 can be formed from a synthetic fabric (for example, a PET fabric) that is shaped set to assume the expanded shape when the prosthetic valve is radially expanded.

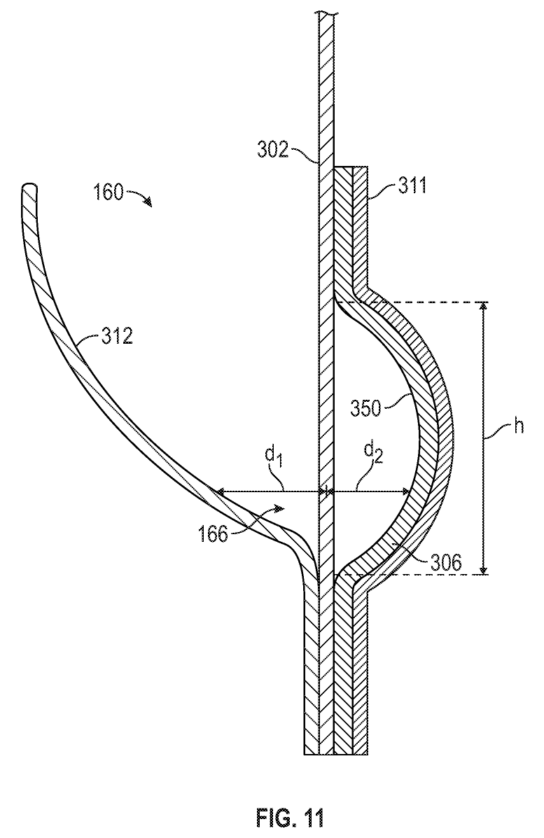

FIG. 11 is a simplified view similar to FIG. 5, also showing how the skirt 306 can be urged radially outward by a radial distance d₂ to expand or enlarge the sinus 160 to a significant degree. The dimension h indicates the approximate height of the open space formed by the radially expanded skirt.

According to another approach, a docking component, which is a separate component from the prosthetic heart valve, can be implanted in the native annulus before the prosthetic heart valve is implanted. Subsequently, the prosthetic heart valve is implanted and preferably received in the docking component. The docking component helps improve blood flow around the leaflets of the prosthetic heart valve in the same general way as discussed above by creating a space between the native leaflets and the prosthetic heart valve, thus reducing the risk of thrombosis. The docking component is fitted with a skirt (or sealing element), which generally replaces the skirt of the prosthetic heart valve (in some cases, the prosthetic heart valve may have a relatively small skirt to assist in other aspects of sealing yet allows retrograde blood to flow radially outwardly through the frame into the neosinus).

In some prosthetic heart valves, the greatest radial dimension of the unexpanded prosthetic heart valve is determined by the radial dimension of the skirt. By using a docking component to support the skirt instead of the prosthetic heart valve, the crimp profile of the prosthetic heart valve can be reduced.

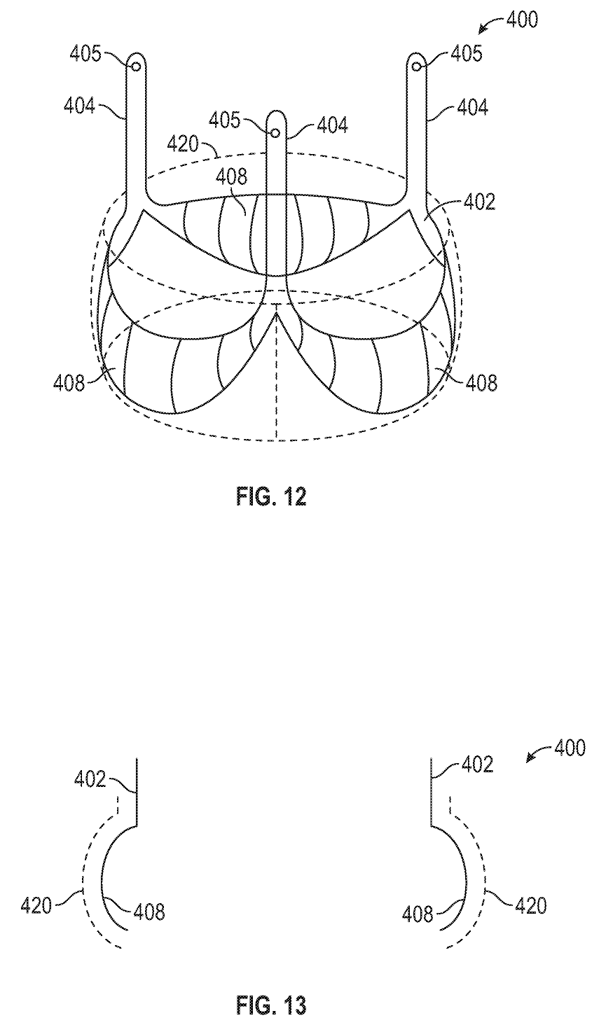

In a first example as shown in FIG. 12, a docking component 400 has a docking component frame 402 that is ring-shaped and a skirt 420 coupled to the outside of the docking component frame 402 (the skirt 420 is shown as transparent to illustrate the docking component frame 402 in more detail). The docking component 400 is provided as a separate component from the prosthetic heart valve and is delivered from the delivery system to the implantation site separately from the prosthetic heart valve. The prosthetic valve 300 and the docking component 400 together form a prosthetic heart valve assembly.

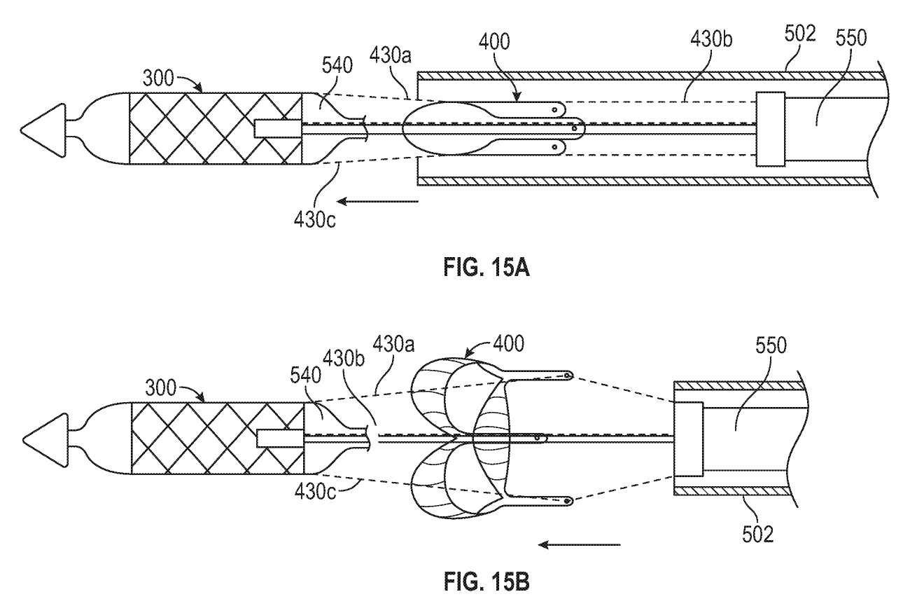

The frame 402 is shown in an expanded state with expanding regions 408 or members that have been reconfigured from an unexpanded state prior to implantation (FIG. 15) to the expanded state (FIGS. 12-14). In the expanded state, the expanding regions 408 urge the skirt 420 radially outward against the native leaflets, similar to the expanding members 350 as described above and shown in FIG. 11.

FIG. 13 is a simplified sectional view of the docking component 400 of FIG. 12, showing the expanding regions 408 and the skirt 420 in the expanded state and urged radially outward at the left and right sides of the illustrated section. As shown, the expanding regions 408 and the skirt 420 have a bulb or bulbous shape when expanded. In some examples, as shown in FIG. 12, the expanding regions 408 collectively form an annular ring having a tri-lobular shape and the skirt 420 assumes a similar tri-lobular shape when the docking component is radially expanded such that the docking component mimics the shape of the native sinus. In FIG. 13, the skirt 420 is shown spaced from the frame 402 for purposes of illustration. However, the skirt 420 can lie against the outer surface of the frame 402.

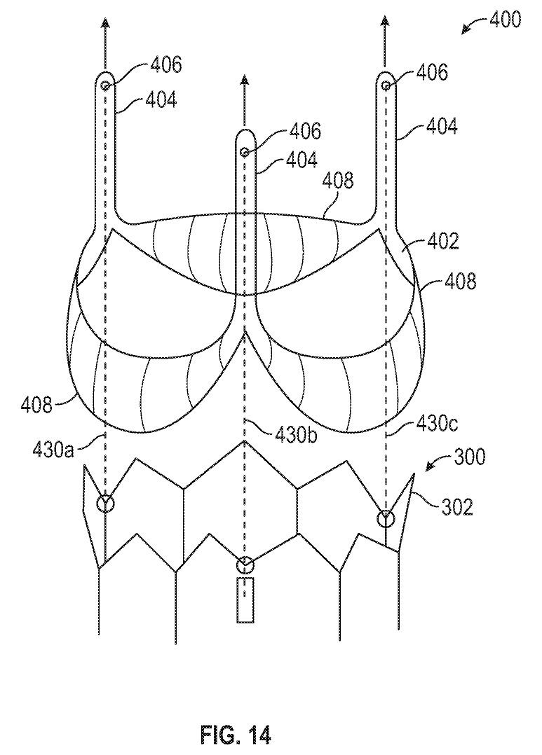

The docking component frame 402 has extension members 404 (also referred to as connection posts) that extend generally axially as shown. Each extension member 404 has an opening 406 (or other guiding structure) near its distal end for forming a releasable connection with a delivery apparatus. The extension members 404 are described in connection with FIG. 14.

FIG. 14 shows the docking component 400 in a stationary position as it would occupy when implanted in the annulus and expanded, with the skirt 420 and the surrounding tissue removed for clarity of illustration. The prosthetic heart valve 300, shown in a partially expanded state, is below and aligned with the central opening in the docking component 400. Sutures 430a, 430b and 430cextend from the prosthetic heart valve 300 (for example, above the commissures), through the central opening in the docking component 400 and through the opening 406 in each respective extension member 404. As is described in more detail below, with the docking component 400 in place, tension applied to the sutures 430a, 430b, 430c is used to move the prosthetic heart valve 300 axially relative to the docking component 400. At the same time, tension in the sutures 430a, 430b, 430c maintains alignment in the circumferential direction (or rotational direction) of the prosthetic heart valve 300 relative to the docking component 400. In this way, the scallop of each leaflet of the prosthetic heart valve is kept in alignment with a corresponding one of the bulbous expanding regions 408 of the docking component 400 as the prosthetic heart valve is moved into place.

FIGS. 15A and 15B are side sectional views showing an example of a delivery system 500 used to deliver the docking component 400 and the prosthetic heart valve 300 for implantation. The delivery system 500 has a tubular sheath 502 that houses the docking component 400 and the prosthetic heart valve 300, as well as other components. In FIG. 15A, the prosthetic heart valve 300 is shown after it has been moved distally away from an initial position within the sheath 502 to a position outside of the sheath 502 as shown. Specifically, a balloon 540 of a balloon catheter has been partially inflated to contact an inside surface of the prosthetic heart valve 300, and the balloon catheter has been moved axially to in turn move the prosthetic heart valve 300 distally. The docking component 400, shown in its crimped state, has been moved distally from an initial position, but as shown is still within the sheath 502. The sutures 430a, 430b and 430c extend distally from a delivery system shaft 550, through the docking component 400 (via the extension members 404) and to the prosthetic heart valve 300.

In FIG. 15B, further axial movement of the shaft 550 has moved the docking component 400 distally beyond the end of the sheath 502, and the docking component 400 has been reconfigured into a deployed state at the implantation site. For example, the docking component can self-expand into a radially expanded, shape-set state as shown.

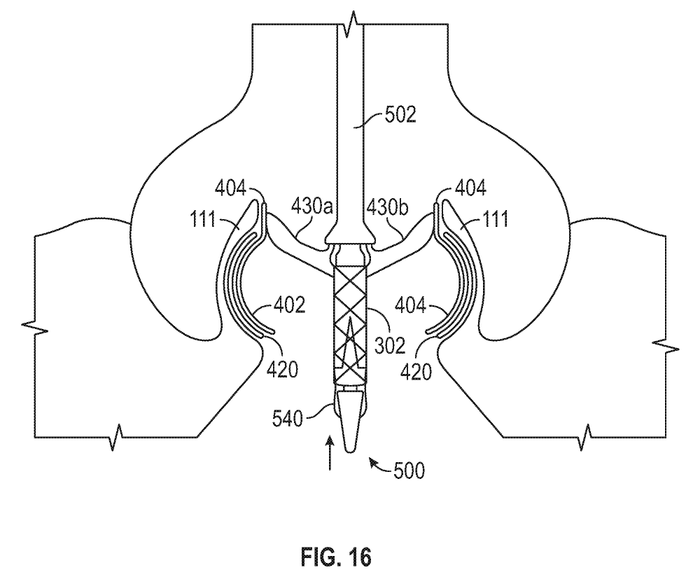

FIGS. 16-18 are schematic cross-sectional views of the delivery system 500 of FIGS. 15A and 15B at subsequent steps of the procedure and showing the native anatomy. In FIG. 16, the docking component 400 is shown implanted in the native aortic annulus in its radially expanded state. Specifically, the docking component frame 402 has been expanded, which in turn has urged the skirt 420 into contact with the native leaflets 110 and into the bulb shape as shown. The sutures 430a and 430b visible in the figure extend from the frame 302 of the prosthetic heart valve 300, through the extension members 404 of the docking component 400, and into the sheath 502 of the delivery system 500. In some examples, the frame 402 is made of a shape memory material (for example, Nitinol), so that the docking component can self-expand from the radially compressed state to the radially expanded state when deployed from the sheath 502.

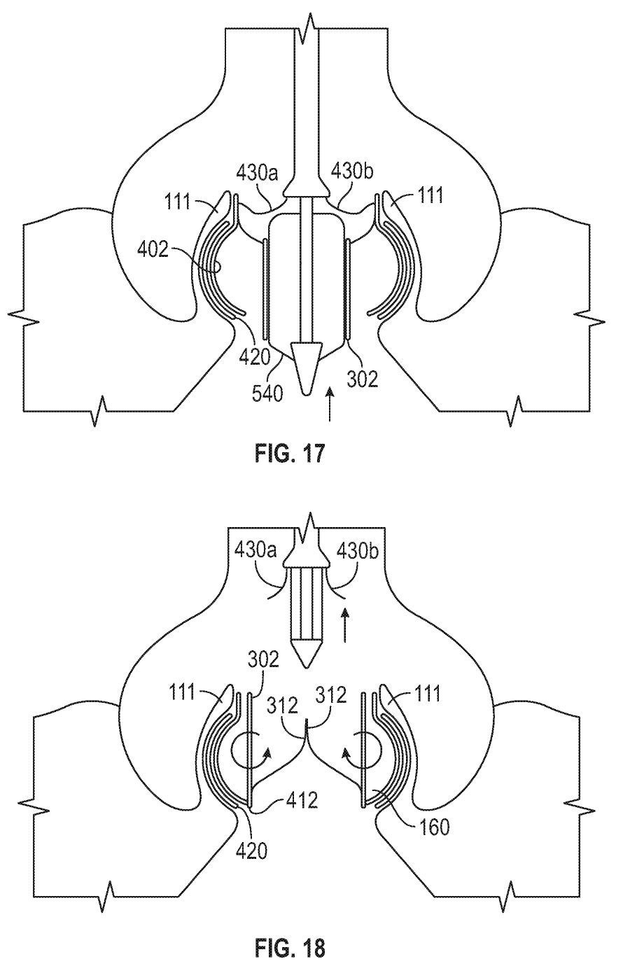

In FIGS. 16 and 17, the prosthetic heart valve 300 is shown being moved axially in the proximal direction (upwardly in the drawing figures) from the position in FIG. 15B towards a final axial position (FIG. 18) aligned within the docking component 400. The prosthetic heart valve 300 is moved axially by moving the balloon catheter proximally or pulling the sutures 430a, 430b and 430c via the shaft 550, or a combination of these or similar movements.

In FIG. 17, the balloon 540 is being inflated further to begin expanding the prosthetic heart valve 300 from its crimped state to its expanded state. The sutures, including the sutures 430a and 430b as shown, are maintained in tension to assist in aligning the prosthetic heart valve 300 rotationally within the docking component 400. In this way, the scallop of each leaflet of the prosthetic heart valve 300 is kept in alignment with a corresponding one the bulbous expanding regions 408 of the docking component 400.

In FIG. 18, the prosthetic heart valve 300 is shown fully expanded in the radial direction, seated, and aligned axially and rotationally within the docking component 400. The prosthetic leaflets 312 are shown in the closed state during diastole. Improved blood flow results because of the expanded space in the neosinus 160 defined between the prosthetic leaflets and the skirt 420. The prosthetic valve 300 in this example does not include any skirts, or alternatively, it may have one or more skirts, but the skirts are sized to allow retrograde blood to flow outwardly through the openings in the frame in the radial direction into space between the prosthetic valve 300 and the docking component 400. The sutures 430a and 430b have been removed from the frame 302 and the extension members 404 (for example, one end of the sutures can be released, and the other end can be pulled through the shaft and an attached handle). With the implantation of the docking component 400 and the prosthetic heart valve 300 completed, the delivery system 500 is being withdrawn from the implantation site.

In other examples, the prosthetic valve 300 and the docking component 400 can be pre-assembled and attached to each other with the prosthetic valve 300 disposed within the docking component before these components are inserted into a patient. For example, the frame 302 of the prosthetic valve can be connected to the frame 402 of the docking component with any of various techniques or mechanisms, such as with sutures, rivets, connecting pins, by welding or an adhesive. In other examples, the frame 302 and the frame 402 can be integrally formed from the same piece of material, such as by laser cutting the frame 302, 402 from a piece of metal (for example, a metal tube).

The skirts (for example, skirts 306, 420) disclosed herein can be wholly or partly formed of any suitable biological material, synthetic material (for example, any of various polymers), or combinations thereof. In some examples, the skirts can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirts can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirts can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the skirts can comprise a sponge material or foam, such as polyurethane foam. In some examples, the skirts can comprise natural tissue, such as pericardium (for example, bovine pericardium, porcine pericardium, equine pericardium, or pericardium from other sources).

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art. Also, any of the delivery techniques described above can be used to deliver a docking component for a prosthetic valve, using the same delivery device (as shown in FIGS. 15-17) or a separate delivery device.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Simulation

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

### Example 1. A prosthetic heart valve comprising:

a valve frame comprising an outflow end and an inflow end, wherein the valve frame is radially expandable from a radially compressed state to a radially expanded state, wherein the valve frame comprises a plurality of struts arranged to form a plurality of cells defining openings in the valve frame;

a plurality of leaflets disposed within and coupled to the valve frame; and

a skirt assembly mounted to an outer surface of the valve frame, wherein the skirt assembly comprises a sealing layer and a plurality of expanding members coupled to the sealing layer, wherein the sealing layer has an inflow edge and an outflow edge secured to the valve frame and an intermediate portion between the inflow edge and the outflow edge, wherein the expanding members are configured to displace the intermediate portion radially outward from the valve frame when the valve frame is expanded to the radially expanded state to enlarge a neosinus defined between the leaflets and the sealing layer;

wherein when the prosthetic heart valve is implanted in a native heart valve and the valve frame is expanded to the radially expanded state, retrograde blood inside the valve frame can flow outwardly through cells of the valve frame and into a region of the neosinus that is between the valve frame and the sealing layer.

Example 2. The prosthetic heart valve of any example herein, particularly example 1, wherein the plurality of expanding members are formed of a shape-memory material.

Example 3. The prosthetic heart valve of any example herein, particularly examples 1-2, wherein the plurality of expanding members are interwoven within the skirt assembly.

Example 4. The prosthetic heart valve of any example herein, particularly examples 1-3, wherein the plurality of expanding members have coupled portions that are coupled to the skirt assembly and free portions that extend between the skirt assembly and the valve frame.

Example 5. The prosthetic heart valve of any example herein, particularly examples 1-4, wherein the plurality of expanding members are oriented generally axially.

Example 6. The prosthetic heart valve of any example herein, particularly examples 1-5, wherein the plurality of expanding members comprises spaced-apart, elongate strands.

Example 7. The prosthetic heart valve of any example herein, particularly examples 1-6, wherein the plurality of expanding members have first ends and second ends opposite the first ends, wherein the first ends are coupled to the skirt assembly adjacent the outflow edge and the second ends are spaced axially from the first ends and towards the inflow edge of the skirt assembly.

Example 8. The prosthetic heart valve of any example herein, particularly example 7, wherein the second ends are coupled to the skirt assembly at second end positions spaced from the inflow edge towards the outflow edge, wherein the expanding members have varying lengths such that the second end positions define a boundary shaped as a repeating curve that generally follows a shape of an inflow edge of the leaflets.

Example 9. The prosthetic heart valve of any example herein, particularly examples 1-8, wherein the expanding members are displaceable to urge the intermediate portion of the skirt assembly radially outward into a bulb-shaped space, and wherein an interior of the bulb-shaped space provides additional volume for blood circulation between the skirt assembly and the plurality of leaflets.

Example 10. A prosthetic heart valve comprising:

a valve frame comprising an outflow end and an inflow end, wherein the valve frame is radially expandable from a radially compressed state to a radially expanded state, wherein the valve frame comprises a plurality of struts arranged to form a plurality of cells defining openings in the valve frame;

a plurality of prosthetic leaflets disposed within and coupled to the valve frame; and

a skirt assembly mounted to an outer surface of the valve frame, wherein the skirt assembly comprises a sealing layer having an inflow edge and an outflow edge, wherein a portion of the sealing layer is configured to be displaced radially outward from the valve frame when the valve frame is expanded to the radially expanded state to enlarge a neosinus defined between the prosthetic leaflets and the sealing layer;

wherein when the prosthetic heart valve is implanted in a native heart valve and the valve frame is expanded to the radially expanded state, retrograde blood inside the valve frame can flow outwardly through cells of the valve frame and into a region of the neosinus that is between the valve frame and the sealing layer.

Example 11. The prosthetic heart valve of any example herein, particularly example claim 10, wherein the skirt assembly further comprises plurality of expanding members coupled to the sealing layer, wherein the expanding members are formed of a shape-memory material.

Example 12. The prosthetic heart valve of any example herein, particularly examples 10-11, wherein the plurality of expanding members have coupled portions that are coupled to the skirt assembly and free portions that extend between the skirt assembly and the valve frame.

Example 13. The prosthetic heart valve of any example herein, particularly examples 11-12, wherein the expanding members are displaceable to urge the skirt assembly radially outward into a bulb-shaped space, and wherein an interior of the bulb-shaped space provides additional volume for blood circulation between the skirt assembly and the plurality of prosthetic leaflets.

Example 14. A prosthetic heart valve assembly comprising:

a docking component that is radially expandable from a radially compressed state to a radially expanded state, the docking component comprising an annular docking frame comprising an inflow end, an outflow end, and an intermediate portion extending axially between the inflow end and the outflow end, wherein a diameter of the intermediate portion is greater than a diameter of the inflow end and a diameter of the outflow end in the radially expanded state, the docking component further comprising an annular sealing layer covering a surface of the docking frame; and

a prosthetic heart valve component, separate from the docking component, that is radially expandable from a radially compressed state to a radially expanded state, the prosthetic heart valve component comprising an annular valve frame and a plurality of leaflets disposed within and coupled to the valve frame, wherein the valve frame comprises a plurality of struts arranged to form a plurality of cells defining openings in the valve frame;

wherein the docking component is configured to be implanted in a native heart valve in its radially expanded state and the prosthetic heart valve component is configured to be implanted in the docking component in a radially expanded state such that a neosinus is defined between the leaflets and the sealing layer that allows retrograde blood inside the valve frame to flow outwardly through cells of the valve frame and into space between the valve frame and the sealing layer.

Example 15. The assembly of any example herein, particularly example 14, wherein the docking frame is self-expandable from its radially compressed state to its radially expanded state.

Example 16. The assembly of any example herein, particularly examples 14-15, wherein the valve frame is plastically deformable from its radially compressed state to its radially expanded state.

Example 17. The assembly of any example herein, particularly examples 14-16, wherein at least one of the docking frame and the valve frame comprises members made of a shape-memory material.

Example 18. The assembly of any example herein, particularly examples 14-17, wherein the docking component comprises extension members that extend axially from the outflow end, the extension members having openings for receiving lengths of suture material.

Example 19. The assembly of any example herein, particularly examples 14-18, wherein the valve frame comprises suture attachment points at which lengths of suture material can be attached.

Example 20. The assembly of any example herein, particularly example 19, wherein the lengths of suture material interconnect the docking component and the valve frame, and wherein the lengths of suture material are tensionable to pull and/or rotate the valve frame relative to the docking component.

Example 21. A method of implanting a prosthetic heart valve and a docking component with a delivery system, comprising:

positioning the docking component at a docking component position on the delivery system, the docking component having an expandable docking component frame and a skirt coupled to an outside of the docking component frame;

positioning the prosthetic heart valve at a prosthetic heart valve position;

advancing the delivery system to an implantation site to align the docking component with a native valve annulus;

radially expanding the docking component frame;

further radially expanding the skirt coupled to the docking component frame against native leaflets;

moving the delivery system to align the prosthetic heart valve with the docking component; and

radially expanding the prosthetic heart valve into contact with the docking component, wherein an enlarged neosinus is defined between an outside of the prosthetic valve and an inside of the skirt of the docking component, the enlarged neosinus promoting increased washout of blood.

The features described herein with regard to any example can be combined with other features described in any one or more of the other examples, unless otherwise stated. For example, any one or more features of one device can be combined with any one or more features of another device.

In view of the many possible ways in which the principles of the disclosure may be applied, it should be recognized that the illustrations depict examples of the disclosed technology and should not be taken as limiting the scope of the disclosure or the claims. Rather, the scope of the claimed subject matter is defined by the following claims and their equivalents.

We claim:

1. A prosthetic heart valve comprising:
a valve frame comprising an outflow end and an inflow end, wherein the valve frame is radially expandable from a radially compressed state to a radially expanded state, wherein the valve frame comprises a plurality of struts arranged to form a plurality of cells defining openings in the valve frame;
a plurality of leaflets disposed within and coupled to the valve frame; and
a skirt assembly mounted to an outer surface of the valve frame, wherein the skirt assembly comprises a sealing layer and a plurality of expanding members coupled to the sealing layer, wherein the plurality of expanding members are formed of a shape-memory material;
wherein the sealing layer has an inflow edge and an outflow edge secured to the valve frame and an intermediate portion between the inflow edge and the outflow edge, wherein the expanding members are configured to displace the intermediate portion radially outward from the valve frame when the valve frame is expanded to the radially expanded state to enlarge a neosinus defined between the leaflets and the sealing layer;
wherein when the prosthetic heart valve is implanted in a native heart valve and the valve frame is expanded to the radially expanded state, retrograde blood inside the valve frame can flow outwardly through cells of the valve frame and into a region of the neosinus that is between the valve frame and the sealing layer.

2. The prosthetic heart valve of claim 1, wherein the plurality of expanding members are interwoven within the skirt assembly.

3. The prosthetic heart valve of claim 1, wherein the plurality of expanding members have coupled portions that are coupled to the skirt assembly and free portions that extend between the skirt assembly and the valve frame.

4. The prosthetic heart valve of claim 1, wherein the plurality of expanding members are oriented generally axially.

5. The prosthetic heart valve of claim 1, wherein the plurality of expanding members comprises spaced-apart, elongate strands.

6. The prosthetic heart valve of claim 1, wherein the plurality of expanding members have first ends and second ends opposite the first ends, wherein the first ends are coupled to the skirt assembly adjacent the outflow edge and the second ends are spaced axially from the first ends and towards the inflow edge of the skirt assembly.

7. The prosthetic heart valve of claim 6, wherein the second ends are coupled to the skirt assembly at second end positions spaced from the inflow edge towards the outflow edge, wherein the expanding members have varying lengths such that the second end positions define a boundary shaped as a repeating curve that generally follows a shape of an inflow edge of the leaflets.

8. The prosthetic heart valve of claim 1, wherein the expanding members are displaceable to urge the intermediate portion of the skirt assembly radially outward into a bulb-shaped space, and wherein an interior of the bulb-shaped space provides additional volume for blood circulation between the skirt assembly and the plurality of leaflets.

9. A prosthetic heart valve comprising:
a valve frame comprising an outflow end and an inflow end, wherein the valve frame is radially expandable from a radially compressed state to a radially expanded state, wherein the valve frame comprises a plurality of struts arranged to form a plurality of cells defining openings in the valve frame;
a plurality of prosthetic leaflets disposed within and coupled to the valve frame; and
a skirt assembly mounted to an outer surface of the valve frame, wherein the skirt assembly comprises a sealing layer having an inflow edge and an outflow edge, wherein the skirt assembly further comprises a plurality of expanding members coupled to the sealing layer, wherein the expanding members are formed of a shape-memory material, and wherein a portion of the sealing layer is configured to be displaced radially outward from the valve frame by the expanding members when the valve frame is expanded to the radially expanded state to enlarge a neosinus defined between the prosthetic leaflets and the sealing layer;
wherein when the prosthetic heart valve is implanted in a native heart valve and the valve frame is expanded to the radially expanded state, retrograde blood inside the valve frame can flow outwardly through cells of the valve frame and into a region of the neosinus that is between the valve frame and the sealing layer.

10. The prosthetic heart valve of claim 9, wherein the plurality of expanding members have coupled portions that are coupled to the skirt assembly and free portions that extend between the skirt assembly and the valve frame.

11. The prosthetic heart valve of claim 9, wherein the expanding members are displaceable to urge the skirt assembly radially outward into a bulb-shaped space, and wherein an interior of the bulb-shaped space provides additional volume for blood circulation between the skirt assembly and the plurality of prosthetic leaflets.

12. A prosthetic heart valve assembly comprising:
a docking component that is radially expandable from a radially compressed state to a radially expanded state, the docking component comprising an annular docking frame comprising an inflow end, an outflow end, and an intermediate portion extending axially between the inflow end and the outflow end, wherein a diameter of the intermediate portion is greater than a diameter of the inflow end and a diameter of the outflow end in the radially expanded state, the docking component further comprising an annular sealing layer covering a surface of the docking frame, and wherein the docking frame comprises a plurality of expanding regions that are expandable to displace the sealing layer radially outward; and
a prosthetic heart valve component, separate from the docking component, that is radially expandable from a radially compressed state to a radially expanded state, the prosthetic heart valve component comprising an annular valve frame and a plurality of leaflets disposed within and coupled to the valve frame, wherein the valve frame comprises a plurality of struts arranged to form a plurality of cells defining openings in the valve frame;
wherein the docking component is configured to be implanted in a native heart valve in its radially expanded state and the prosthetic heart valve component is configured to be implanted in the docking component in a radially expanded state such that a neosinus is defined between the leaflets and the sealing layer that allows retrograde blood inside the valve frame to flow outwardly through cells of the valve frame and into space between the valve frame and the sealing layer.

13. The assembly of claim 12, wherein the docking frame is self-expandable from its radially compressed state to its radially expanded state.

14. The assembly of claim 12, wherein the valve frame is plastically deformable from its radially compressed state to its radially expanded state.

15. The assembly of claim 12, wherein at least one of the docking frame and the valve frame comprises members made of a shape-memory material.

16. The assembly of claim 12, wherein the docking component comprises extension members that extend axially from the outflow end, the extension members having openings for receiving lengths of suture material.

17. The assembly of claim 12, wherein the valve frame comprises suture attachment points at which lengths of suture material can be attached.

18. The assembly of claim 17, wherein the lengths of suture material interconnect the docking component and the valve frame, and wherein the lengths of suture material are tensionable to pull and/or rotate the valve frame relative to the docking component.

19. A method of implanting a prosthetic heart valve and a docking component with a delivery system, comprising:
positioning the docking component at a docking component position on the delivery system, the docking component having an expandable docking component frame and a skirt coupled to an outside of the docking component frame;
positioning the prosthetic heart valve at a prosthetic heart valve position;
advancing the delivery system to an implantation site to align the docking component with a native valve annulus;
radially expanding the docking component frame;
further radially expanding the skirt coupled to the docking component frame against native leaflets via expandable regions of the docking component frame that displace the skirt radially outward;
moving the delivery system to align the prosthetic heart valve with the docking component; and
radially expanding the prosthetic heart valve into contact with the docking component, wherein an enlarged neosinus is defined between an outside of the prosthetic valve and an inside of the skirt of the docking component, the enlarged neosinus promoting increased washout of blood.
